(19) United States
(12) Patent Application Publication
(10) Pub. No.: US 2026/0125685 A1
(43) Pub. Date: May 7, 2026

(54) THERAPEUTIC AGENT FOR TREATING HIF-1alpha-MEDIATED DISEASES BY INHIBITING AREL1 AND RESTORING PHD2 ACTIVITY

(71) Applicant: BIOCHEMGEN INC., Cheonan-si Chungcheongnam-do (KR)

(72) Inventor: Deug-Yong SHIN, Yongin-si Gyeonggi-do (KR)

(21) Appl. No.: 19/440,674

(22) Filed: Jan. 6, 2026

Related U.S. Application Data
(63) Continuation-in-part of application No. PCT/KR2024/006090, filed on May 7, 2024.

(30) Foreign Application Priority Data
Jul. 12, 2023 (KR) 10-2023-0090751

(51) Int. Cl. C12N 15/113; A61K 45/06; A61P 35/00; C12N 15/10; C12N 15/88; C12Q 1/48; G01N 33/573

(52) U.S. Cl. CPC C12N 15/1137; A61K 45/06; A61P 35/00; C12N 15/1055; C12N 15/88; C12Q 1/48; G01N 33/573; C12N 2310/14; C12N 2310/531; C12Y 203/02; G01N 2333/9108; G01N 2333/91177

(57) ABSTRACT

Provided are agents and methods for treating or preventing diseases mediated by pathological HIF-1α activity. The disclosure provides an AREL1 inhibitor that restores PHD2 stability or activity and thereby decreases HIF-1α protein level and/or transcriptional activity. In exemplary embodiments, inhibition of AREL1 decreases expression of HIF-1α target genes such as VEGF and suppresses angiogenesis, supporting use of the disclosed agents for treating cancer and other angiogenesis-driven disorders. In some embodiments, the diseases include autoimmune and inflammatory diseases.

### INCORPORATION OF SEQUENCE LISTING

This application contains a sequence listing submitted in Computer Readable Form (CRF). The CRF file containing the sequence listing entitled “PK002597882-SequenceListing.xml”, which was created on Jan. 2, 2026, and is 2034 bytes in size. The information in the sequence listing is incorporated herein by reference in its entirety.

### TECHNICAL FIELD

The present disclosure relates to therapeutic and preventive agents for diseases associated with pathological activation of hypoxia-inducible factor-1 alpha (HIF-1α). More particularly, the disclosure relates to agents that inhibit apoptosis-resistant E3 ubiquitin protein ligase 1 (AREL1), or that inhibit an interaction between AREL1 and prolyl hydroxylase domain-containing protein 2 (PHD2), thereby increasing PHD2 stability or activity and reducing HIF-1α activity. The agents are useful, for example, for treating cancers and other angiogenesis-driven disorders.

### BACKGROUND ART

HIF-1α is a transcription factor that enables cellular adaptation to hypoxia. Under normoxia, HIF-1a is hydroxylated by PHD2 and subsequently recognized by the von Hippel-Lindau (VHL) ubiquitin ligase complex, leading to proteasomal degradation. Under hypoxia, PHD2 activity is reduced, HIF-1α accumulates, and HIF-1α activates expression of hypoxia-responsive genes, including genes involved in angiogenesis, metabolism, invasion, and survival.

Pathological activation of HIF-1α has been implicated in tumor growth, therapy resistance, metastasis, and abnormal angiogenesis. Accordingly, there is a need for therapeutic strategies that reduce HIF-1α activity in diseases where HIF-1α is aberrantly elevated. Pathological HIF-1α activation has also been implicated in immune dysregulation and chronic inflammation, including autoimmune and inflammatory diseases such as rheumatoid arthritis, psoriasis, inflammatory bowel disease, and autoimmune thyroid diseases.

AREL1 (also known as RANI1 in earlier literature) is an E3 ubiquitin ligase previously associated with anti-apoptotic signaling. However, the role of AREL1 in regulating the PHD2-HIF-1α axis and angiogenesis has not been fully elucidated. The present disclosure identifies AREL1 as a regulator that promotes HIF-1α accumulation by targeting PHD2 for ubiquitination and degradation, and provides therapeutic approaches based on inhibiting AREL1 to restore PHD2 function and suppress HIF-1α activity.

### DISCLOSURE

### Technical Problem

An object of the present disclosure is to provide agents and methods for treating or preventing diseases mediated by pathological HIF-1α activity, including cancers and other angiogenesis-driven disorders, by suppressing HIF-1α activity through modulation of the AREL1-PHD2-HIF-1a pathway. In some embodiments, the diseases include cancer and non-cancer HIF-1α-related diseases, including autoimmune and inflammatory diseases in which pathological HIF-1α signaling contributes to disease progression.

Another object is to provide agents that inhibit expression or activity of AREL1, or that inhibit an interaction between AREL1 protein and PHD2 protein, thereby stabilizing PHD2 and reducing HIF-1α protein level and/or transcriptional activity.

The objects of the present disclosure are not limited to those described above, and other objects will be apparent to those skilled in the art from the following description.

### Technical Solution

In one aspect, provided is a pharmaceutical composition comprising, as an active ingredient, an AREL1 inhibitor and a pharmaceutically acceptable carrier.

In some embodiments, the HIF-1α-related disease comprises an autoimmune or inflammatory disease selected from rheumatoid arthritis, psoriasis, alopecia areata, type 1 diabetes, Graves' disease, Hashimoto's thyroiditis, vitiligo, Crohn's disease, and ulcerative colitis. In some embodiments, the HIF-1α-related disease comprises cancer, including solid tumors characterized by hypoxia, angiogenesis, and elevated HIF-1α activity.

As used herein, an “AREL1 inhibitor” includes any agent that (i) reduces expression of AREL1, (ii) inhibits enzymatic activity of AREL1 (including E3 ubiquitin ligase activity), and/or (iii) inhibits binding between AREL1 protein and PHD2 protein.

Non-limiting examples of AREL1 inhibitors include inhibitory nucleic acids (e.g., siRNA, shRNA, antisense oligonucleotides, microRNA, ribozymes), antibodies or antigen-binding fragments that specifically bind AREL1 and inhibit its function, peptides that competitively inhibit the AREL1-PHD2 interaction, aptamers, and small-molecule compounds identified by screening.

In certain embodiments, the inhibitory nucleic acid targets AREL1 mRNA. In a specific embodiment, the inhibitory nucleic acid comprises the sequence: AATTGGTCCCTGAGAACCTTT (SEQ ID NO:1), or a sequence having at least 90% identity thereto and retaining AREL1 knockdown activity. SEQ ID NO:1: AATTGGTCCCTGAGAACCTTT (shRNA/siRNA target sequence against AREL1) In another aspect, provided is a method of treating a HIF-1α-mediated disease in a subject, comprising administering to the subject a therapeutically effective amount of an AREL1 inhibitor or a pharmaceutical composition thereof.

In certain embodiments, the HIF-1α-mediated disease is cancer, including a solid tumor characterized by elevated HIF-1α activity and/or angiogenesis. In additional embodiments, the disease is another angiogenesis-driven disorder in which reduction of HIF-1α activity is beneficial.

The pharmaceutical composition may be formulated for oral, parenteral, intranasal, pulmonary, topical, transdermal, or other routes of administration. Formulations may include, without limitation, tablets, capsules, granules, powders, syrups, solutions, suspensions, injections, emulsions, and controlled-release preparations. Delivery systems such as liposomes, lipid nanoparticles, and emulsions may be used depending on the nature of the active ingredient.

A therapeutically effective amount may vary depending on factors such as subject age, weight, condition, disease severity, administration route, and formulation. Appropriate dosing regimens may be determined by a clinician based on standard pharmacological principles.

### Advantageous Effects

According to the present disclosure, inhibition of AREL1 restores PHD2 stability or activity, thereby decreasing HIF-1α protein level and/or transcriptional activity. As a result, expression of HIF-1α target genes such as VEGF is reduced, leading to suppression of angiogenesis.

Accordingly, the disclosed agents and methods are useful for treating cancers and other disorders mediated by pathological HIF-1α signaling.

The effects of the disclosure are not limited to those described above, and other effects will be apparent to those skilled in the art.

### DESCRIPTION OF DRAWINGS

FIG. 1 illustrates increased HIF-1α protein level upon AREL1 expression.

FIG. 2 illustrates decreased PHD2 protein level and increased HIF-1α protein level with increasing AREL1 expression.

FIG. 3 illustrates increased expression of HIF-1α target genes upon AREL1 expression.

FIG. 4 illustrates increased PHD2 and decreased HIF-1α and VEGF upon inhibition of AREL1.

FIG. 5 illustrates selective binding of AREL1 to PHD family proteins as determined by a GST pull-down assay, wherein a GST-tagged AREL1 construct (for example, an N-terminal half comprising about residues 1-450) pulls down PHD1 and PHD2 under the assay conditions and does not detectably pull down PHD3, and further illustrates that an N-terminal region of PHD1 (e.g., about residues 1-150) is important for the interaction. In some embodiments, AREL1 comprises a C-terminal catalytic region (for example, about the C-terminal ˜450 amino acids) that includes a HECT domain.

FIG. 6 illustrates increased ubiquitination of PHD2 mediated by AREL1.

FIG. 7 illustrates regulation of angiogenesis by AREL1 and inhibition of angiogenesis by AREL1 knockdown.

### BEST MODE

Hereinafter, embodiments of the disclosure are described in detail with reference to the following Examples. These Examples are provided for illustrative purposes only and are not intended to limit the scope of the disclosure.

### Example 1: Increase in HIF-1a by AREL1 Expression

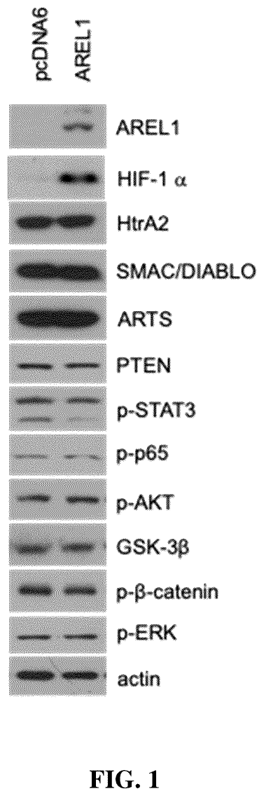

A stable cell line expressing AREL1 was generated and compared to a control cell line. Total protein was extracted and analyzed by immunoblotting. Compared to controls, cells expressing AREL1 exhibited increased HIF-1α protein level (FIG. 1), indicating that AREL1 positively regulates HIF-1α.

### Example 2: Dose-Dependent Decrease in PHD2 and Increase in HIF-1α Caused by AREL1

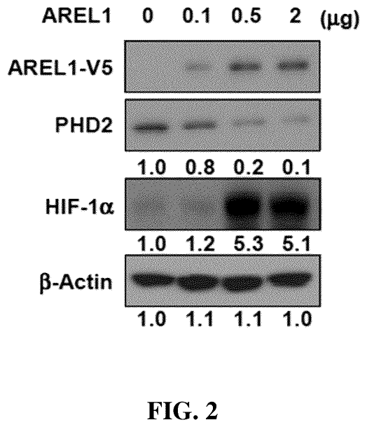

To further evaluate regulation of the PHD2-HIF-1α pathway by AREL1, an AREL1 expression vector was transiently introduced into cells at increasing doses. PHD2 and HIF-1α protein levels were then assessed by immunoblotting. As AREL1 expression increased, PHD2 protein level decreased, while HIF-1α protein level increased (FIG. 2). These results support a model in which AREL1 activates HIF-1α by downregulating PHD2.

### Example 3: Changes in HIF-1α Target Genes Upon AREL1 Modulation

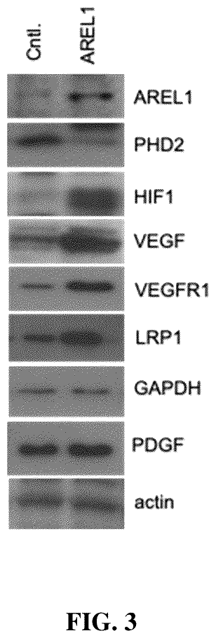

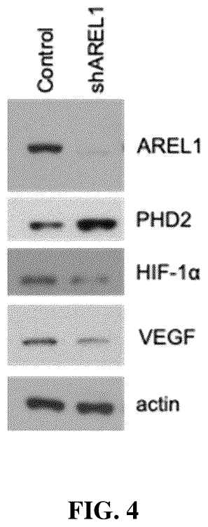

Cells were infected with retroviral vectors expressing AREL1 or an shRNA targeting AREL1 (shAREL1). In cells expressing shAREL1, AREL1 expression was reduced, and PHD2 protein level increased. Consistent with restored PHD2 activity, HIF-1α and HIF-1α target genes (including VEGF) were decreased relative to control cells (FIG. 3 and FIG. 4). These results demonstrate that inhibiting AREL1 suppresses the HIF-1α transcriptional program.

### Example 4: Binding Between AREL1 and PHD2

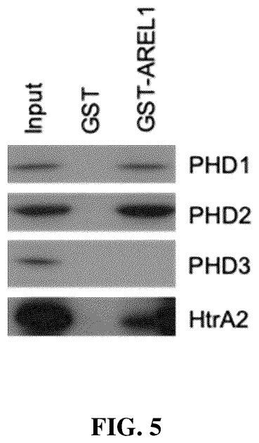

Because PHD2 hydroxylates HIF-1α and promotes its degradation, and because AREL1 expression increased HIF-1α, physical interaction between AREL1 and PHD2 was investigated. Cells were co-transfected with expression vectors encoding AREL1 and PHD2. Co-immunoprecipitation analysis demonstrated that AREL1 protein binds to PHD2 protein (FIG. 5), suggesting that AREL1 may directly regulate PHD2 stability or function.

As shown in FIG. 5, a glutathione S-transferase (GST) pull-down assay using a GST-tagged AREL1 construct (for example, an N-terminal half comprising about residues 1-450) indicated that PHD1 and PHD2 were pulled down with GST-AREL1 under the assay conditions, whereas PHD3 was not detectably pulled down. These results support selective recognition of PHD1/PHD2 relative to PHD3. Domain mapping further indicated that an N-terminal region of PHD1 (for example, about the first 150 amino acids) is important for the interaction. Without being bound by theory, these observations are consistent with the interaction depending on a determinant present in PHD1 and PHD2 but absent in PHD3.

In some embodiments, a yeast two-hybrid (Y2H) format is used to assess binding using an AREL1 fragment. For example, an N-terminal half of AREL1 (e.g., about residues 1-450) is used as a bait construct in Y2H screening to identify interacting partners, including PHD1 and/or PHD2, while showing reduced or no interaction with PHD3.

### Example 5: AREL1-Mediated Ubiquitination of PHD2

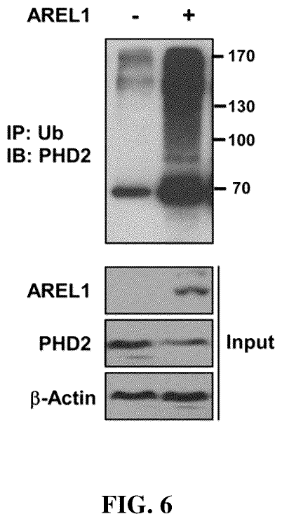

To determine whether AREL1 promotes ubiquitination of PHD2, cells were transfected with expression vectors for PHD2 and AREL1, together with a ubiquitin expression construct. Following immunoprecipitation, ubiquitinated PHD2 was detected by immunoblotting. AREL1 expression increased ubiquitination of PHD2 (FIG. 6), consistent with a mechanism in which AREL1 promotes PHD2 degradation and thereby elevates HIF-1α.

### Example 6: Inhibition of Angiogenesis by AREL1 Knockdown

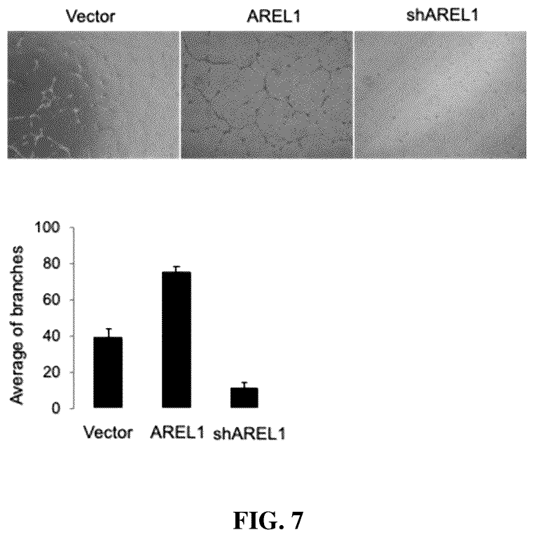

To evaluate the functional consequence of AREL1 modulation on angiogenesis, conditioned media from cells expressing AREL1 or shAREL1 were applied to HUVEC cells seeded on a matrix suitable for tube formation. Tube formation was quantified as a measure of angiogenic activity. Compared with controls, conditioned media from AREL1-expressing cells increased tube formation, whereas conditioned media from shAREL1 cells markedly reduced tube formation (FIG. 7). These results indicate that inhibiting AREL1 suppresses HIF-1α-driven angiogenesis.

### Example 7: Screening Protocol for Identifying Inhibitors of Interaction Between AREL1 and PHD2

To identify candidate agents that inhibit the interaction between AREL1 and PHD2 (and thereby restore PHD2 stability or activity and suppress HIF-1α signaling), test agents may be evaluated using a tiered screening workflow. The following protocol is provided as a non-limiting example, and equivalent assay formats may be used.

### 7-1. Primary Protein-Protein Interaction Assay (PPI Assay)

(a) In one embodiment, cells are engineered to express epitope-tagged AREL1 and epitope-tagged PHD2. Cell lysates are prepared under conditions preserving protein-protein interactions. Lysates are contacted with a test agent, and the AREL1-PHD2 complex is quantified by co-immunoprecipitation (co-IP) followed by immunoblotting or immunoassay detection. (b) In another embodiment, purified AREL1 protein and PHD2 protein (or binding domains thereof) are used in a homogeneous assay format such as AlphaScreen, TR-FRET, BRET, or fluorescence polarization. A decrease in binding signal relative to a control indicates inhibition of the AREL1-PHD2 interaction. In some embodiments, the PPI assay uses full-length AREL1 or an AREL1 fragment such as an N-terminal half (e.g., about residues 1-450) as a bait or binding partner to focus on the interaction interface, while the C-terminal HECT catalytic region may be omitted in the binding construct.

### 7-2. Orthogonal Confirmation and Counterscreens

Primary hits may be confirmed using an orthogonal binding assay such as surface plasmon resonance (SPR), microscale thermophoresis (MST), or biolayer interferometry (BLI). To reduce false positives, counterscreens may be performed to exclude assay interference (e.g., signal quenching, aggregation, non-specific binding) and to confirm that the test agent does not non-specifically disrupt unrelated protein complexes.

In some embodiments, orthogonal confirmation further distinguishes (i) agents that bind AREL1 and disrupt AREL1-PHD2 binding from (ii) agents that bind a binding determinant within an N-terminal region of PHD2 and thereby disrupt AREL1-PHD2 binding. For example, target-binding assays (e.g., SPR, MST, BLI, or thermal shift) may be performed using purified AREL1, purified PHD2, and/or an N-terminal fragment of PHD2. In some embodiments, PHD3 is used as a negative selectivity control such that candidates are prioritized when they disrupt binding to PHD1/PHD2 while showing reduced or no binding to PHD3 under comparable conditions.

### 7-3. Cell-Based Functional Validation (PHD2 Stabilization and HIF-1α Suppression)

Confirmed candidates may be evaluated in cells under normoxia and/or hypoxia (or a hypoxia-mimetic condition) to determine functional consequences. Non-limiting readouts include: (i) increased PHD2 protein level measured by immunoblotting or immunoassay; (ii) reduced ubiquitination of PHD2 measured by immunoprecipitation of ubiquitinated proteins followed by detection of PHD2; (iii) reduced HIF-1α protein level measured by immunoblotting; and/or (iv) reduced HIF-1α transcriptional activity measured using an HIF-responsive element (HRE) reporter assay. Additional downstream validation may include reduced expression of HIF-1α target genes (e.g., VEGF, CA9, GLUT1) measured by qPCR or immunoassay.

### 7-4. Exemplary Selection Criteria

In some embodiments, a candidate agent is advanced when it inhibits the AREL1-PHD2 interaction by at least about 30% to 70% in a primary PPI assay at a screening concentration, shows a dose-dependent effect in an orthogonal assay, maintains acceptable cell viability in a cytotoxicity counterscreen, and reduces HIF-1α transcriptional activity and/or HIF-1α target gene expression in a cell-based assay. Selection thresholds may be adjusted depending on assay format and intended therapeutic modality.

### Example 8 (Prophetic): Identification and Validation of a Candidate AREL1-PHD2 Interaction Inhibitor

This prophetic example describes one way to practice the screening workflow described above. The following results are illustrative and are not intended to represent actual experimental data.

A diverse test library comprising small molecules and/or peptides is screened in a homogeneous AREL1-PHD2 PPI assay at a single concentration. Test agents that reduce binding signal relative to a vehicle control are designated primary hits. Primary hits are re-tested in a concentration-response format to estimate potency and are further evaluated using an orthogonal binding assay (e.g., SPR or MST).

A confirmed candidate agent is then evaluated in a cell-based assay. Cells are treated with the candidate agent under hypoxia or a hypoxia-mimetic condition. Following treatment, PHD2 protein level is assessed and is observed to increase relative to control, consistent with reduced AREL1-mediated ubiquitination or degradation. In parallel, HIF-1α protein level and/or HIF-1α transcriptional activity (HRE reporter) is assessed and is observed to decrease relative to control. Downstream, expression of one or more HIF-1α target genes (for example, VEGF) is assessed and is observed to be reduced.

In additional prophetic experiments, the candidate agent is evaluated for functional consequences relevant to angiogenesis. For example, conditioned media from treated cells is applied to endothelial cells in a tube formation assay, and angiogenic tube formation is reduced relative to control, consistent with suppression of HIF-1α-driven pro-angiogenic signaling.

The disclosure described above is exemplary. Those skilled in the art will appreciate that various modifications and equivalent embodiments are possible. Accordingly, the scope of the disclosure is defined by the appended claims and includes all modifications, equivalents, and alternatives within the spirit and scope of the claims.

### Example 9 (Prophetic): Treatment of an Autoimmune or Inflammatory Disease by Inhibiting AREL1 and Suppressing HIF-1α Signaling

This prophetic example describes one non-limiting approach to evaluate the disclosed AREL1 inhibitors in an autoimmune or inflammatory context. The following results are illustrative and are not intended to represent actual experimental data.

Primary immune cells (for example, macrophages or T cells) or disease-relevant tissue cells (for example, synovial fibroblasts) are cultured under hypoxia or a hypoxia-mimetic condition to induce HIF-1α signaling. Cells are treated with an AREL1 inhibitory nucleic acid or an agent that inhibits the AREL1-PHD2 interaction.

Following treatment, PHD2 protein level is assessed and is observed to increase relative to control. HIF-1α protein level and/or HIF-1α transcriptional activity (HRE reporter) is assessed and is observed to decrease relative to control. In addition, expression of one or more HIF-1α target genes (for example, VEGF, CA9, or GLUT1) is assessed and is observed to be reduced.

In further prophetic studies, the AREL1 inhibitor is evaluated in an animal model of autoimmune or inflammatory disease (for example, a model of arthritis or colitis). Administration of the AREL1 inhibitor is observed to reduce pathological HIF-1α signaling in affected tissues and to improve one or more disease-relevant endpoints relative to control.

1. A method of treating a HIF-1α-related disease in a subject in need thereof, comprising administering to the subject a therapeutically effective amount of an inhibitory nucleic acid that inhibits expression of apoptosis-resistant E3 ubiquitin protein ligase 1 (AREL1), thereby increasing a level or activity of prolyl hydroxylase domain-containing protein 2 (PHD2) and decreasing hypoxia-inducible factor-1 alpha (HIF-1α) activity in cells of the subject.

2. The method of claim 1, wherein the inhibitory nucleic acid is selected from siRNA, shRNA, antisense oligonucleotide, and microRNA.

3. The method of claim 1, wherein the inhibitory nucleic acid comprises AATTGGTCCCTGAGAACCTTT (SEQ ID NO:1), or a sequence having at least 90% identity thereto and retaining AREL1 knockdown activity.

4. The method of claim 1, wherein administering comprises delivering the inhibitory nucleic acid in a lipid nanoparticle (LNP) formulation or a liposome formulation.

5. The method of claim 1, wherein administering comprises systemic administration.

6. The method of claim 1, wherein decreasing HIF-1α activity comprises decreasing HIF-1α transcriptional activity measured by an HIF-responsive element (HRE) reporter assay.

7. The method of claim 1, wherein the HIF-1α-related disease is an autoimmune or inflammatory disease selected from rheumatoid arthritis, psoriasis, alopecia areata, type 1 diabetes, Graves' disease, Hashimoto's thyroiditis, vitiligo, Crohn's disease, and ulcerative colitis.

8. The method of claim 7, wherein the autoimmune or inflammatory disease is rheumatoid arthritis.

9. The method of claim 7, further comprising administering a second therapeutic agent selected from corticosteroids, immunomodulators, anti-cytokine biologics, and small-molecule immunosuppressants.

10. The method of claim 1, wherein the HIF-1α-related disease is cancer.

11. A method of treating a HIF-1α-related disease in a subject in need thereof, comprising administering to the subject a therapeutically effective amount of an agent that inhibits an interaction between AREL1 protein and PHD2 protein, thereby increasing PHD2 protein level and decreasing HIF-1α activity in cells of the subject.

12. The method of claim 11, wherein the agent is selected from a small molecule, a peptide, an antibody, and an inhibitory nucleic acid.

13. The method of claim 11, wherein the agent reduces ubiquitination of PHD2 in cells.

14. The method of claim 11, wherein decreasing HIF-1α activity comprises decreasing HIF-1α transcriptional activity measured by an HRE reporter assay.

15. The method of claim 11, wherein the method decreases expression of a HIF-1α target gene selected from VEGF, CA9, and GLUT1.

16. A pharmaceutical composition comprising (i) an AREL1 inhibitor and (ii) a pharmaceutically acceptable carrier, wherein the AREL1 inhibitor is configured to increase PHD2 level or activity and decrease HIF-1α activity in a cell.

17. The pharmaceutical composition of claim 16, wherein the AREL1 inhibitor is an inhibitory nucleic acid that inhibits expression of AREL1.

18. The pharmaceutical composition of claim 16, wherein the AREL1 inhibitor is an agent that inhibits an interaction between AREL1 protein and PHD2 protein.

19. The pharmaceutical composition of claim 16, wherein the composition is formulated as an LNP formulation for systemic administration.

20. A kit comprising the pharmaceutical composition of claim 16 and instructions for use to treat a HIF-1α-related disease by increasing PHD2 and decreasing HIF-1α activity.

21. The method of claim 11, wherein the agent is identified by a screening method comprising contacting (i) a GST-tagged AREL1 polypeptide comprising an N-terminal half of AREL1 of about residues 1-450 with (ii) PHD2 protein or a polypeptide comprising an N-terminal region of PHD2, and selecting a test agent that reduces binding of PHD2 to the GST-tagged AREL1 polypeptide in a GST pull-down assay.

22. The method of claim 21, wherein the screening method further comprises a yeast two-hybrid assay using the N-terminal half of AREL1 of about residues 1-450 as a bait construct.

23. The method of claim 21, wherein the screening method further comprises confirming, using PHD3 as a negative selectivity control, that the test agent does not cause detectable pull-down of PHD3 by the GST-tagged AREL1 polypeptide under comparable assay conditions.

24. The method of claim 21, wherein the agent binds to the N-terminal region of PHD2 and thereby interferes with binding of PHD2 to AREL1.


SEQUENCE LISTING (1 sequence)

SEQ ID NO: 1 - DNA, 21 bp, synthetic construct
  source 1..21: mol_type = other DNA
aattggtccctgagaaccttt